(19) United States
(12) Patent Application Publication
(10) Pub. No.: US 2010/0055187 A1
(43) Pub. Date: Mar. 4, 2010

(54) NANOVITAMIN SYNTHESIS

(76) Inventor: Dong June Ahn, Seoul (KR)

Correspondence Address: EDWARDS ANGELL PALMER & DODGE LLP, P.O. BOX 55874, BOSTON, MA, 02205, US

(21) Appl. No.: 12/200,748

(22) Filed: Aug. 28, 2008

(51) Int. Cl. A61K 9/14

(52) U.S. Cl. 424/489

(57) ABSTRACT

Stable nanoparticulate vitamin compositions are prepared from agglomerated or larger sized vitamin particles of at least one vitamin compound by breaking down and/or solubilizing the agglomerated or larger sized vitamin particles and associating the particles with a surface modifying agent.

### BACKGROUND

A vitamin is commonly defined as an organic compound required as a nutrient in small or trace amounts by an organism. Ingestion of vitamins as part of the diet is typically necessary because vitamins cannot be synthesized in sufficient quantities by an organism. Thus, the term is conditional both on the circumstance and the particular organism. For example, ascorbic acid functions as vitamin C for some animals but not others, and vitamins D and K are required in the human diet only in certain circumstances.

Vitamins are classified by their biological and chemical activity, not their structure. Thus, each “vitamin” actually refers to a number of vitamer compounds, which form a set of distinct chemical compounds that show the biological activity of a particular vitamin. Each set of chemicals is grouped under an alphabetized vitamin “generic descriptor” title, such as “vitamin A,” which, for example, includes retinal, retinol, and many carotenoids. Vitamers are often inter-convertible in the body. The term vitamin does not include other essential nutrients such as dietary minerals, essential fatty acids, or essential amino acids, nor does it encompass the large number of other nutrients that promote health but are otherwise required less often.

Vitamins have diverse biochemical functions. Some vitamins function as hormones (e.g., vitamin D), antioxidants (e.g., vitamin E), and mediators of cell signaling and regulators of cell and tissue growth and differentiation (e.g., vitamin A). Many vitamins (e.g., B complex vitamins) function as precursors for enzyme cofactor bio-molecules (coenzymes) that help act as catalysts and substrates in metabolism. When acting as part of a catalyst, vitamins are bound to enzymes and are called prosthetic groups. For example, biotin forms part of enzymes involved in making fatty acids. Vitamins also act as coenzymes to carry chemical groups between enzymes. For example, folic acid carries various carbon groups (e.g., methyl, formyl, and methylene) in the cell.

Until the 1900s, vitamins were obtained solely through food intake, therefore changes in diet can alter the types and amounts of vitamins ingested. For example, as the availability of certain foods changes according to the seasons, dietary patterns change and the ingestion of vitamins changes. In recent times, vitamins have been produced chemically and made widely available as inexpensive pills, allowing supplementation of the dietary intake.

While the definition of “vitamin” is somewhat fluid, there are 13 dietary substances that are generally recognized as vitamins in the human diet. Substances generally accepted to be vitamins and their corresponding vitamers include, but are not limited to, vitamin A (retinol, retinal retinoids, and carotenoids), vitamin B₁ (thimine), vitamin B₂ (riboflavin), Vitamin B₃ (niacin, niacinamide), vitamin B₅ (pantothenic acid), vitamin B₆ (pyridoxine, pyridoxamine, pyridoxal), vitamin B₇ (biotin), vitamin B₉ (folic acid, folinic acid), vitamin B₁₂ (cyanocobalimin, hydroxycobalamin, methylcobalamin), vitamin C (ascorbic acid), vitamin D (ergocalciferol, cholecalciferol), vitamin E (tocopherols, tocotrienols), and vitamin K (phylloquinone, menaquinones).

Vitamins are classified as either water-partitionable, meaning that they dissolve easily in water, or lipid-partitionable, which are typically soluble in most common organic solvents and are absorbed through the intestinal tract with the help of lipids. In general, water-partitionable vitamins are readily excreted from the body, while lipid-partitionable vitamins are retained for a longer period of time. Water-partitionable vitamins include the B vitamins (i.e., vitamins B₁, B₂, B₃, B₅, B₆, B₇, B₉, and B₁₂) and vitamin C. Lipid-partitionable vitamins include vitamins A, D, E and K.

### BRIEF SUMMARY

The illustrated embodiments relate to novel stable nanoparticulate vitamin compositions and methods for manufacturing the same. The stable nanoparticulate vitamin compositions are prepared by starting with agglomerated or larger sized vitamin particles of at least one vitamin compound and suspending them in at least one solvent in the presence of at least one surface modifying agent to form a slurry. The at least one solvent is typically selected such that the at least one vitamin compound is practically insoluble therein. Stable nanoparticulate vitamin compositions are formed by breaking down and/or solubilizing the agglomerated or larger sized vitamin particles and associating the particles with the surface modifying agent.

The illustrated embodiments are based partly on the discovery that vitamin particles having a small effective particle size can be prepared by breaking down larger vitamin particles in the presence of a solvent in which the vitamin particles are not soluble in conjunction with a surface modifier. Such particles are stable and do not appreciably flocculate or agglomerate due to interparticle attractive forces and can be formulated into vitamin supplement compositions exhibiting unexpectedly high bioavailability. Their greater bioavailability means, for example, that nanoparticulate vitamin compositions can be given in smaller doses with less of the vitamins passing through the body unabsorbed.

In one embodiment, a method of preparing a stable nanoparticulate vitamin composition is described. In one embodiment, the method includes providing a precursor mixture that includes particles of the at least one vitamin compound, at least one solvent in which the at least one vitamin compound has a solubility of less than 10 mg/ml, and molecules of at one surface modifying agent; and treating the precursor mixture to produce smaller sized vitamin particles, wherein the molecules of the at least one surface modifying agent stably associate with and stabilize the smaller sized vitamin particles.

In one embodiment, the vitamin particles in the precursor mixture are provided as a powder or slurry of individual particles or agglomerates having a size in a range of about 100 μm to about 2 μm, or about 75 μm to about 3 μm, or about 50 μm to about 4 μm.

In one embodiment, the treating step of the method described above further includes transferring the precursor mixture to a microfluidizer having an interaction chamber capable of producing shear, impact, cavitation, and attrition forces; and subjecting the precursor mixture to said forces at a temperature not exceeding 40° C. and a fluid pressure of from about 3,000 to about 30,000 psi by passing the precursor mixture through said interaction chamber to obtain vitamin particles having an effective average particle size in a range from about 1 nm to about 2000 nm. One will appreciate that it may take multiple passes through the microfluidizer in order to obtain vitamin particles having the desired size.

In another embodiment, the treating step of the method described above further includes sonicating the precursor mixture a temperature not exceeding 40° C., for a time in a range from about 5 minutes to about 2 hours, wherein the sonicating suspends the particles of the at least one vitamin in the solvent, allows the surface modifying agent to associate with the particles of the at least one vitamin, and disrupts or breaks down the particles of the at least one vitamin into smaller vitamin particles having a second size in a desired size range.

The particles obtained following the microfluidizer treatment or the sonication treatment are typically substantially sphere-shaped and/or substantially rod-shaped.

In a broad range, the vitamin particles in the stable nanoparticulate vitamin composition have a size in a range from about 1 nm to about 2000 nm. In a narrower range, the vitamin particles in the stable nanoparticulate vitamin composition have a size in a range from about 50 nm to about 1500 nm. In a still narrower range, the vitamin particles in the stable nanoparticulate vitamin composition have a size in a range from about 100 nm to about 1000 nm.

In one embodiment, the method further includes selecting at least one water-partitionable vitamin compound or at least one lipid-partitionable vitamin compound. Water-partitionable vitamin compounds may be chosen from the group consisting of vitamin B1, vitamin B2, vitamin B3, vitamin B5, vitamin B₆, vitamin B7, vitamin B9, vitamin B12, or vitamin C, and combinations thereof. Lipid-partitionable vitamin compounds may be chosen from the group consisting of vitamin A, vitamin D, vitamin E, or vitamin K, and combinations thereof.

In one embodiment, the solvent is selected from the group consisting of water, aqueous salt solutions, methanol, ethanol, propanol, butanol, glycerol, propylene glycol, propylene glycol ethers, dimethyl formamide, N-methyl pyrrolidone, acetone, diethyl ether, chloroform, benzene, tetrahydrofuran, hexanes, ethyl acetate, methyl methacrylate, toluene, phenyl ethers, vegetable oil, and combinations thereof.

In one embodiment the solvent may be selected for compatibility with water-partitionable vitamin compounds or lipid-partitionable vitamin compounds. That is, the solvent may be selected such that the at least one vitamin compound has a solubility of less than 10 mg/ml in the selected solvent. Suitable examples of solvents in which water-partitionable vitamin compounds have a solubility of less than 10 mg/ml include, but are not limited to, acetone, diethyl ether, chloroform, benzene, tetrahydrofuran, hexanes, ethyl acetate, methyl methacrylate, toluene, phenyl ethers, vegetable oils (e.g., safflower oil or rape seed oil), and combinations thereof. Suitable examples of solvents in which lipid-partitionable vitamin compounds have a solubility of less than 10 mg/ml include, but are not limited to, water, aqueous salt solutions, methanol, ethanol, propanol, butanol, glycerol, propylene glycol, or propylene glycol ethers, and combinations thereof.

Suitable examples of surface modifying agents include, but are not limited to, at least one of an organic acid, a long-chain amine, a surfactant, an anionic surface stabilizer, a cationic surface stabilizer, a zwitterionic surface stabilizer, or an ionic surface stabilizer, and optionally includes one or more long-chain alcohols.

In one embodiment, a stable nanoparticulate vitamin composition is described. The stable nanoparticulate vitamin composition includes nano-particles of at least one vitamin compound having a size in a range from about 1 nm to about 2000 nm and molecules of at least one stabilizing agent associated with the nano-particles.

In one embodiment, the nano-particles of at least one vitamin compound are selected from a group consisting of water-partitionable vitamins or a group consisting of lipid-partitionable vitamins.

In one embodiment, the stable nanoparticulate vitamin composition further includes at least one solvent in which the at least one vitamin compound has a solubility of less than 10 mg/ml. Suitable examples of solvents include, but are not limited to, water, aqueous salt solutions, methanol, ethanol, propanol, butanol, glycerol, propylene glycol, propylene glycol ethers, dimethyl formamide, N-methyl pyrrolidone, acetone, diethyl ether, chloroform, benzene, tetrahydrofuran, hexanes, ethyl acetate, methyl methacrylate, toluene, phenyl ethers, vegetable oil, and combinations thereof.

These and other objects and features of nanoparticulate vitamin compositions will become more fully apparent from the following description and appended claims, or may be learned by the practice of the claims as set forth hereinafter. The foregoing summary is illustrative only and is not intended to be in any way limiting.

### DETAILED DESCRIPTION

The illustrative embodiments described in the detailed description and claims are not meant to be limiting. Other embodiments may be utilized, and other changes may be made, without departing from the spirit or scope of the subject matter presented here.

### I. Introduction

The illustrated embodiments relate to novel stable nanoparticulate vitamin compositions and methods for manufacturing the same. The stable nanoparticulate vitamin compositions are prepared by starting with agglomerated or larger sized vitamin particles of at least one vitamin compound and suspending them in at least one solvent in the presence of at least one surface modifying agent to form a slurry. The at least one solvent is typically selected such that the at least one vitamin compound is practically insoluble therein. Stable nanoparticulate vitamin compositions are formed by breaking down the agglomerated or larger sized vitamin particles and associating the particles with the surface modifying agent.

The illustrated embodiments are based partly on the discovery that vitamin particles having a small effective particle size can be prepared by breaking down larger vitamin particles in the presence of a solvent in which the vitamin particles are not soluble in conjunction with a surface modifier. Such particles are stable and do not appreciably flocculate or agglomerate due to interparticle attractive forces and can be formulated into vitamin supplement compositions exhibiting unexpectedly high bioavailability. For example, their greater bioavailability means that nanoparticulate vitamin compositions can be given in smaller doses with less of the vitamins passing through the body unabsorbed.

As used herein, the term “nanoparticulate compositions” refers to stabilized nano-scale particles of a therapeutic or diagnostic agent having a coating of a stabilizing surface modifying agent. In some instances, “nanoparticulate compositions” include a solvent that suspends the stabilized particles.

As used herein, the term “vitamin particles” refers to solid, crystalline phase particles of various vitamin vitamer compounds, such as but not limited to, vitamin B₁₂, which is typically provided as cyanocobalimin, hydroxycobalamin, or methylcobalamin.

As used herein, the term “precursor mixture” refers to a mixture of compounds used to make a stable nanoparticulate vitamin composition. In a minimal sense, the precursor mixture includes a plurality of vitamin particles, at least one solvent, and at least on stabilizing compound. The vitamin particles may be provided as a powder or a slurry.

As used herein, the term “stable” or “stably suspended” when used in the context of a stable nanoparticulate vitamin composition refers to a system in which particles of between 1 nm and 2000 nm are suspended or dispersed in a continuous phase of a different composition (i.e., a solvent) such that the stabilized nanoparticles do not appreciably fall out of suspension and/or agglomerate over a relatively long period of time (e.g., weeks or months).

As used herein, the “surface modifying agent” refers to a compound or mixture of compounds that are compatible with a given solvent and that associate with the surface of vitamin particles to prevent coagulation or agglomeration of the particles in stable suspension.

As used herein, the term “nano-scale” or “nano-sized” means a size between about 1 nm and about 2000 nm.

II. Components used to Manufacture Stable Nanoparticulate Vitamin Compositions

The following components can be used to carry out methods for manufacturing stable nanoparticulate vitamin compositions of vitamin particles according to the illustrated embodiments.

### A. Vitamin Compounds

The vitamin compounds used to prepare the stable nanoparticulate vitamin compositions according to the illustrated embodiments are provided as powders of individual particles and/or agglomerates or as solvent-based slurries of individual particles and/or agglomerates. Examples of suitable vitamin particles that can be used in the illustrated embodiments include, but are not limited to, vitamin A, vitamin B₁, vitamin B₂, vitamin B₃, vitamin B₅, vitamin B₆ (pyridoxine, pyridoxamine, pyridoxal), vitamin B₇ (biotin), vitamin B₉ (folic acid, folinic acid), vitamin B₁₂ (cyanocobalimin, hydroxycobalamin, methylcobalamin), vitamin C (ascorbic acid), vitamin D (ergocalciferol, cholecalciferol), vitamin E (tocopherols, tocotrienols), and vitamin K (phylloquinone, menaquinones). The vitamin substance can be present in an essentially pure or crystalline form.

In a broad range, the vitamin particles in the stable nanoparticulate vitamin composition have a size in a range from about 1 nm to about 2000 nm. In a narrower range, the vitamin particles in the stable nanoparticulate vitamin composition have a size in a range from about 50 nm to about 1500 nm. In a still narrower range, the vitamin particles in the stable nanoparticulate vitamin composition have a size in a range from about 100 nm to about 1000 nm. In some embodiments, the vitamin particles have a size in a range from about 1 nm, 5 nm, 10 nm, 50 nm, 100 nm, 250 nm, 500 nm, 750 nm, 1000 nm, or 1500 nm, to about 5 nm, 10 nm, 50 nm, 100 nm, 250 nm, 500 nm, 750 nm, 1000 nm, 1500 nm, or 2000 nm. In some embodiments, the vitamin particles have a size of about 1 nm, 5 nm, 10 nm, 50 nm, 100 nm, 250 m, 500 nm, 750 nm, 1000 nm, 1500 nm, or 2000 nm.

Vitamin A is a lipid partitionable vitamin important in vision and bone growth. Vitamers of vitamin A include, but are not limited to, retinol, retinal retinoids, and carotenoids. Retinol is ingested in a precursor form; animal sources (e.g., liver and eggs) contain retinyl esters, whereas plants (e.g., carrots, spinach) contain pro-vitamin A carotenoids. Hydrolysis of retinyl esters results in retinol while pro-vitamin A carotenoids can be cleaved to produce retinal, which can be reversibly reduced to produce retinol or it can be irreversibly oxidized to produce retinoic acid.

Vitamin B₁ is a water-soluble B-complex vitamin important for neural function and carbohydrate metabolism. The most common vitamer of vitamin B₁ is thiamin. It is soluble in water, methanol, and glycerol and practically insoluble in acetone, ether, chloroform, and benzene. Thiamin is found in a wide variety of many foods at low concentrations, with yeast, liver and cereal grains being the most common

Vitamin B₂ is a water-soluble B-complex vitamin that is an important component of the cofactors FAD and FMN required by all flavoproteins. Vitamin B₂ plays a key role in energy metabolism, and is required for the metabolism of fats, ketone bodies, carbohydrates, and proteins. The most common vitamer of vitamin B₂ is riboflavin. Milk, cheese, leafy green vegetables, liver, kidneys, legumes such as mature soybeans, yeast, almonds and some shellfish are good sources of vitamin B₂.

Vitamin B₃ is a water-soluble B-complex vitamin that is a precursor for the enzyme co-factors NADH, NAD, NAD+, and NADP, which play essential metabolic roles in living cells, DNA repair, and the production of steroid hormones in the adrenal gland. The most common vitamers of vitamin B₃ include, but are not limited to, niacin and niacinamide. Most animal- and plant-based foods are rich sources of vitamin B₃.

Vitamin B₅ is a water-soluble B-complex vitamin needed to form coenzyme-A (CoA), and is critical in the metabolism and synthesis of carbohydrates, proteins, and fats. The most common vitamer of vitamin B₅ is pantothenic acid. Small quantities of pantothenic acid are found in nearly every food, with high amounts in whole-grains, legumes, eggs, and meat.

Vitamin B₆ is a water-soluble B-complex vitamin that is the precursor for pyridoxal phosphate (PLP). PLP is a cofactor in many reactions of amino acid metabolism, including transamination, deamination, and decarboxylation. PLP also is necessary for the enzymatic reaction governing the release of glucose from glycogen. The most common vitamers of vitamin B₆ include, but are not limited to, pyridoxine, pyridoxamine, pyridoxal. Vitamin B₆ is widely distributed in foods in both its free and bound forms. Good sources of vitamin B₆ include meats, whole grain products, vegetables, and nuts.

Vitamin B₇ is a water-soluble B-complex vitamin that acts as a cofactor in the metabolism of fatty acids and leucine, and in gluconeogenesis. The most common vitamer of vitamin B₇ is biotin. The most important natural sources of biotin in human nutrition are milk, liver, egg (egg yolk), and some vegetables.

Vitamin B₉ is a water-soluble B-complex vitamin necessary for the production and maintenance of new cells. This is especially important during periods of rapid cell division and growth such as infancy and pregnancy. Folate is needed to synthesize DNA bases (most notably thymine, but also purine bases) needed for DNA replication. The most common vitamers of vitamin B₉ are folic acid and folinic acid. Leafy vegetables such as spinach, turnip greens, lettuces, dried beans and peas, fortified cereal products, sunflower seeds and certain other fruits and vegetables are rich sources of vitamin B₉.

Vitamin B₁₂ is a water-soluble B-complex vitamin that is important for the normal functioning of the brain and nervous system, and for the formation of blood. It is normally involved in the metabolism of every cell of the body, especially affecting DNA synthesis and regulation, but also fatty acid synthesis and energy production. The most common vitamers of vitamin B₁₂ include, but are not limited to, cyanocobalimin, hydroxycobalamin, and methylcobalamin. Cyanocobalimin does not generally occur naturally, but it is the most common vitamer of B₁₂ in dietary supplements because it is more air stable than the other vitamers. Vitamin B₁₂ is naturally found in meat (especially liver and shellfish), milk and eggs. Animals, in turn, must obtain it directly through food or indirectly from bacteria that inhabit the gut.

Vitamin C is a water-soluble vitamin that acts as an antioxidant and is a cofactor in several enzymatic reactions. The most common vitamer of vitamin C is ascorbic acid or ascorbate ion. Many plant-based and animal-based foods are rich sources of vitamin C.

Vitamin D is a lipid partitionable vitamin that is important for many bodily functions including regulating calcium and phosphorus levels in the blood, promoting bone formation, immune system regulation. The most common vitamers of vitamin D include, but are not limited to, ergocalciferol and cholecalciferol. Cholecalciferol is produced in skin exposed to sunlight, specifically ultraviolet B radiation. Many foods are rich sources of vitamin D, including supplemented dairy products.

Vitamin E is a collective name for a set of 8 related tocopherols and tocotrienols, which are fat-soluble vitamins with important antioxidant properties. Many foods such as asparagus, avocado, and fish oils are rich sources of vitamin E.

Vitamin K denotes a group of lipophilic, hydrophobic vitamins that are needed for the posttranslational modification of certain proteins, mostly required for blood coagulation. The most common vitamers of vitamin K include, but are not limited to, phylloquinone, menaquinones. Vitamin K is found leafy green vegetables, such as spinach and kale, cabbage, cauliflower, broccoli, brussels sprouts, and some fruits, such as avocado and kiwifruit.

### B. Solvents

The solvents used to prepare the stable nanoparticulate vitamin compositions provide a continuous phase for dispersing vitamin particles of the precursor mixture and/or dispersing the vitamin particles of the stable nanoparticulate vitamin compositions. The solvent serves as a carrier for the vitamin particles and the surface modifying agent. Various solvents or mixtures of solvents can be used, including but not limited to, water and organic solvents.

The vitamin substance is typically poorly soluble and dispersible in at least one liquid solvent. By “poorly soluble” it is meant that the vitamin substance has a solubility in the liquid dispersion medium, e.g., water, of less than about 10 mg/ml, or less than about 1 mg/ml.

The choice of solvent is at least partly a function of vitamin substance or substances. Vitamins are typically classified as either water-partitionable, meaning that they dissolve easily in water, or lipid-partitionable, meaning that they dissolve easily in most common organic solvents and are absorbed through the intestinal tract with the help of lipids. Water-partitionable vitamins include, but are not limited to, the B complex vitamins (i.e., vitamins B₁, B₂, B₃, B₅, B₆, B₇, B₉, and B₁₂) and vitamin C. Lipid-partitionable vitamins include, but are not limited to, vitamins A, D, E and K.

Suitable examples of solvents in which water-partitionable vitamin compounds have a solubility of less than 10 mg/ml include, but are not limited to, acetone, diethyl ether, chloroform, benzene, tetrahydrofuran, hexanes, ethyl acetate, methyl methacrylate, toluene, phenyl ethers, vegetable oils (e.g., safflower oil or rape seed oil), and combinations thereof.

Suitable examples of solvents in which lipid-partitionable vitamin compounds have a solubility of less than 10 mg/ml include, but are not limited to, water, aqueous salt solutions, methanol, ethanol, propanol, butanol, glycerol, propylene glycol, or propylene glycol ethers, and combinations thereof.

### C. Surface Modifying Agents

The surface modifying agents used to prepare the stable nanoparticulate vitamin compositions associate with the surface of vitamin particles to prevent coagulation or agglomeration of the particles by overcoming the tendency of vitamin particles to agglomerate due to, e.g., inter-particle attraction. A surface modifying agent or a mixture of agents is selected such that it is dispersible or otherwise compatible with a given solvent used to form the stable nanoparticulate vitamin composition. For example, the agent or agents can be weakly solublized by the solvent so that the surface modifying agent is free to bond to and/or associate with the vitamin particles, but the solvent does not tend to wash the molecules of surface modifying agent off of the vitamin particles.

Molecules of the surface modifying agent molecules are complexed with the vitamin particles to control formation of the stable nanoparticulate vitamin composition. The surface modifying agent is selected to promote the formation of nanoparticulate vitamin particles that have a desired stability, size, and/or uniformity. Examples of suitable surface modifying agents include, but are not limited to, a variety of organic molecules, polymers, and oligomers. The surface modifying agent can interact and bond with the vitamin particles dissolved or dispersed within an appropriate solvent or carrier through various mechanisms, including ionic bonding, covalent bonding, lone pair electron bonding, hydrogen bonding, or van der Waals forces. In one embodiment, useful surface modifiers are believed to include those which physically adhere to the surface of the vitamin particle but do not chemically bond to the vitamin.

Suitable surface modifiers can preferably be selected from known organic and inorganic pharmaceutical excipients. Such excipients include, but are not limited to, various polymers, low molecular weight oligomers, natural products and surfactants. Preferred surface modifiers include nonionic and anionic surfactants. Representative examples of excipients include gelatin, casein, lecithin (phosphatides), gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, e.g., macrogol ethers such as cetomacrogol 1000, polyoxyethylene castor oil derivatives, polyoxyethylene sorbitan fatty acid esters, e.g., the commercially available Tweens, polyethylene glycols, polyoxyethylene stearates, colloidol silicon dioxide, phosphates, sodium dodecylsulfate, carboxymethylcellulose calcium, carboxymethylcellulose sodium, methylcellulose, hyclroxyethylcellulose, hydroxypropylcellulose, hydroxypropylmethycellulose phthalate, noncrystalline cellulose, magnesium aluminum silicate, triethanolamine, polyvinyl alcohol (PVA), and polyvinylpyrrolidone (PVP). Most of these excipients are described in detail in the Handbook of Pharmaceutical Excipients, published jointly by the American Pharmaceutical Association and The Pharmaceutical Society of Great Britain, the Pharmaceutical Press, 1986. The surface modifiers are commercially available and/or can be prepared by techniques known in the art. Two or more surface modifiers can be used in combination.

Other examples of suitable surface modifiers include, but are not limited to, organic acids, long-chain amines, and surfactants. In addition to an organic acid, a long-chain amine, and/or a surfactant, the surface modifying agent may optionally include at least one long-chain alcohol.

Examples of suitable organic acids include so-called fatty acids. A fatty acid is an organic compound with a carboxylic acid head group and an aliphatic tail. The tail may be either saturated or unsaturated. A saturated fatty acid has no double bonds in its tail (i.e., the tail is fully saturated with hydrogen). An unsaturated fatty acid has at least one double bond in its tail (i.e., the tail is not fully saturated with hydrogen).

Examples of suitable saturated fatty acids include, but are not limited to, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, uncosanoic acid, docosanoic acid, tricosanoic acid, and tetracosanoic acid. This series of fatty acids have tail lengths that range from four carbons to 24 carbons. In some embodiments, metal salts of the fatty acids may be used in lieu of or in addition to the carboxylic acid form.

Examples of suitable unsaturated fatty acids include, but are not limited to, undecylenic acid, myristoleic acid, palmitoleic acid, oleic acid, linoleic acid, alpha-linolenic acid, arachidonic acid, eicosapentaenoic acid, erucic acid, and docosahexaenoic acid. This series of fatty acids have tail lengths that range from 11 carbons to 24 carbons. In some embodiments, metal salts of the fatty acids may be used in lieu of or in addition to the carboxylic acid form.

Examples of suitable long-chain amines include, but are not limited to, butylamine, pentylamine, hexylamine, heptylamine, octylamine, nonylamine, decylamine, undecylamine, dodecylamine, tridecylamine, tetradecylamine, pentadecylamine, hexadecylamine, heptadecylamine, octadecylamine, nonadecylamine, eicosylamine, uncosylamine acid, docosylamine acid, tricosylamine acid, tetracosylamine, decenylamine, undecenylamine, dodecenylamine, tridecenylamine, tetradecenylamine, pentadecenylamine, hexadecenylamine, heptadecenylamine, octadecenylamine, nonadecenylamine, eicocenylamine, uncocenylamine, dococenylamine, tricocenylamine, and tetracocenylamine.

Examples of suitable surfactants include, but are not limited to, octylphenol ethoxylates, phosphonic acids, phosphinic acids, sulfonic acids, and polyethylene glycol monoalkyl ethers. Example octylphenol ethoxylates include detergents of the well-known Triton-X series. Examples of Triton-X detergents include Triton-X 15, Triton-X 35, Triton-X 45, Triton-X 100, Triton-X 102, Triton-X 114, Triton-X 165, Triton-X 305, Triton-X 405, and Triton-X 705. Polyethylene glycol monoalkyl ethers have the general formula CH₃(CH₂)_{y}O(CH₂CH₂O)ₓH. Example polyethylene glycol monoalkyl ethers include tetraethylene glycol monooctyl ether, pentaethylene glycol monooctyl ether, hexaethylene glycol monooctyl ether, pentaethylene glycol monodecyl ether, pentaethylene glycol monodecyl ether, nonaethylene glycol monodecyl ether, octaethylene glycol monododecyl ether, nonaethylene glycol monododecyl ether, decaethylene glycol monododecyl ether, octaethylene glycol monotridecyl ether, and dodecyl glycol monodecyl ether.

Examples of suitable long-chain alcohols are organic compounds with at least one hydroxyl functional group attached to an aliphatic tail. The aliphatic tail may be unbranched or branched and the aliphatic tail may be saturated or unsaturated. Examples of long-chain alcohols include, but are not limited to, butanol, isobutanol, pentanol, hexanol, heptanol, octanol, nonanol, decanol, undecanol, dodecanol, tetradecanol, cetyl alcohol, stearyl alcohol, arachidyl alcohol, docosanol, octanosol, ethyl hexanol, palmitoleyl alcohol, stearyl alcohol, isostearyl alcohol, elaidyl alcohol, oleyl alcohol, linoleyl alcohol, elaidolinoleyl alcohol, linolenyl alcohol, elaidolinolenyl alcohol, ricinoleyl alcohol, arachidyl alcohol, behenyl alcohol, erucyl alcohol, lignoceryl alcohol, ceryl alcohol, montanyl alcohol, myricyl alcohol, lacceryl alcohol, geddyl alcohol, 1-hexadecanol, 1-octadecanol, 1-eicosanol, 1-docosanol, 1-tetracosanol, 1-hexacosanol, 1-octacosanol, 1-triacontanol, 1-dotriacontanol, and 1-tetratriacontanol.

Additional examples of suitable surface modifiers include, but are not limited to, cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oils, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hydroxypropyl methylcellulose, carboxymethylcellulose sodium, methylcellulose, hydroxyethylcellulose, hydroxypropylmethyl-cellulose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-derivatized phospholipid, PEG-derivatized cholesterols, PEG-derivatized vitamin A, PEG-derivatized vitamin E, random copolymers of vinyl acetate and vinyl pyrrolidone, a polymer, a biopolymer, a polysaccharide, a cellulosic, an alginate, a nonpolymeric compound, a phospholipid, zwitterionic stabilizers, poly-n-methylpyridinium, anthryul pyridinium chloride, chitosan, polylysine, polyvinylimidazole, polybrene, polymethylmethacrylate trimethylammoniumbromide bromide (PMMTMABr), hexyldesyltrimethylammonium bromide (HDMAB), polyvinylpyrrolidone-2-dimethylaminoethyl methacrylate dimethyl sulfate, 1,2 Dipalmitoyl-sn-Glycero-3-Phosphoethanolamine-N-[Amino(Polyethylene Glycol)2000] (sodium salt), Poly(2-methacryloxyethyl trimethylammonium bromide), poloxamines, lysozyme, alginic acid, carrageenan, POLYOX, cationic lipids, sulfonium, phosphonium, quarternary ammonium compounds, stearyltrimethylammonium chloride, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium bromide, C₁₂₋₁₅dimethyl hydroxyethyl ammonium chloride, C₁₂₋₁₅dimethyl hydroxyethyl ammonium bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl(ethenoxy)₄ ammonium chloride, lauryl dimethyl(ethenoxy)₄ ammonium bromide, N-alkyl(C₁₂₋₁₈)dimethylbenzyl ammonium chloride, N-alkyl(C₁₄₋₁₈)dimethyl-benzyl ammonium chloride, N-tetradecyldimethylbenzyl ammonium chloride monohydrate, dimethyl didecyl ammonium chloride, N-alkyl and (C₁₂₋₁₄)dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄)dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride, dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, polyquaternium 10, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, quaternized ammonium salt polymers, alkyl pyridinium salts, amines, protonated quaternary acrylamides, methylated quaternary polymers, cationic guar, benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3) oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

### III. Manufacturing Stable Nanoparticulate Vitamin Compositions

The stable nanoparticulate vitamin compositions are prepared via a method that may include one or more steps such as, but not limited to, (1) selecting at least one vitamin compound, (2) providing a precursor mixture that includes particles of the at least one vitamin compound, at least one solvent in which the at least one vitamin compound has a solubility of less than 10 mg/ml, and a surface modifying agent that is dispersible in the at least one solvent and is capable of stably associating with the particles of the at least one vitamin compound, and (3) treating the precursor mixture to produce the stable nanoparticulate vitamin composition, wherein the stable nanoparticulate vitamin composition includes smaller sized vitamin particles (e.g., than is the precursor mixture) having a coating of surface modifying agent.

In the treating step of the method the vitamin particles are broken down to form a stable nanoparticulate vitamin composition. In practice, the treating step is carried out using, e.g., a microfluidizer and/or a sonicator.

### A. The Microfluidizer

The primary forces attributed to microfluidization by the microfluidizer for producing either emulsions or dispersions, and for reducing mean particle size include, but are not limited to:

shear, involving boundary layers, turbulent flow, acceleration and change in flow direction;

impact, involving collision of the particles processed with solid elements of the microfluidizer, and collision between the particles being processed; and

cavitation, involving an increased change in velocity with a decreased change in pressure, and turbulent flow.

An additional force can be attributed to attrition, i.e., grinding by friction.

A typical microfluidizer consists of an air motor connected to a hydraulic pump which circulates the process fluid. The formulation stream is propelled at high pressures (up to e.g. 23,000 psi) through a specially designed interaction chamber which has fixed microchannels that focus the formulation stream and accelerate it to a high velocity. Within the chamber the formulation is subjected to intense shear, impact and cavitation, all of which contribute to particle size reduction. After processing, the formulation stream is passed through a heat exchanger coil and can be collected or recirculated through the machine. A microfluidizer is typically used in a continuous processing mode for up to three hour of total processing time. The heat exchanger and interaction chamber are externally cooled with a refrigerated circulating water bath.

### B. Sonication

Sonication acts to break down the vitamin particles in the precursor mixture to smaller particles primarily through inducing high velocity interparticle collisions in the slurry and through the formation of microbubbles that generate violent shockwaves and microjets when the bubbles collapse. The force of interparticle collisions is a function of solvent type and the intensity of the sonic energy that is transmitted into the slurry. Bubble collapse and the forces generated therein are a function of the solvent type and the temperature of the solvent during sonication. Briefly stated, the forces generated by bubble collapse are greatest if the vapor pressure of the solvent inside the bubble is minimized. Vapor pressure is a function of solvent type and temperature. As such, it can be advantageous to sonicate at a temperature in a range from about 0° C. to about 60° C., or in a range from about 1° C. to about 40° C., or in a range from about 2° C. to about 20° C., or in a range from about 5° C. to about 10° C.

### C. The Process of Making the Nanoparticulates

Regardless of the procedure utilized, e.g., microfluidization or sonication or a blend of the two, the coarse vitamin substance is selected and added to a liquid medium in which it is essentially insoluble to form a premix. The concentration of the vitamin substance in the liquid medium can range from about 0.1 wt % to about 60 wt %. It is typical, but not essential, that the surface modifier be present in the premix. In some embodiments, concentration of the vitamin substance in the liquid medium can range from about 0.1 wt %, 0.2 wt %, 0.5 wt %, 1 wt %, 5 wt %, 10 wt %, 15 wt %, 20 wt %, 25 wt %, 30 wt %, 35 wt %, 40 wt %, 45 wt %, 50 wt %, 55 wt %, or about 60 wt %.

The concentration of the surface modifier can vary from about 0.1 to 90%, or from about 1-75%, or from about 20-60%, by weight based on the total combined weight of the vitamin substance and surface modifier. In some embodiments, the concentration of the surface modifier can vary from about 0.1%, 0.2%, 0.5%, 1%, 5%, 10%, 15%, 20%, 25%, 30%, 35%, 40%, 45%, 50%, 55%, 60%, 65%, 70%, 75%, 80%, 85%, or 90%, to about 1%, 5%, 10%, 15%, 20%, 25%, 30%, 35%, 40%, 45%, 50%, 55%, 60%, 65%, 70%, or 75%, or to about 20%, 25%, 30%, 35%, 40%, 45%, 50%, 55%, or 60%.

The relative amount of vitamin substance and surface modifier can vary widely and the optimal amount of the surface modifier can depend, for example, upon the particular vitamin substance and surface modifier selected, the critical micelle concentration of the surface modifier if it forms micelles, etc.

The premix can be circulated in a microfluidizer continuously first at low pressures, then at maximum capacity having a fluid pressure of from about 3,000 to 30,000 psi until the desired particle size reduction is achieved. The particles should be reduced in size at a temperature which does not significantly degrade the vitamin substance. Processing temperatures in a range from about 0° C. to about 60° C. are typical. Processing temperature in a range from about 1° C. to about 40° C., or in a range from about 2° C. to about 20° C., or in a range from about 5° C. to about 10° C. can also be usefully employed.

There are at least two methods to collect a slurry and re-pass it in a microfluidizer. The “discreet pass” method collects every pass through the microfluidizer until all of the slurry has been passed through before re-introducing it again to the microfluidizer. This guarantees that every substance or particle has “seen” the interaction chamber the same amount of times. A second method recirculates the slurry by collecting it in a receiving tank and allowing the entire mixture to randomly mix and pass through the interaction chamber. We have found that recirculating a slurry is just as effective as the “discreet pass” method, however, maintaining slurry homogeneity in the receiving tank is important.

In the case of sonication, the precursor mixture is sonicated for a period of time sufficient to break down the vitamin particles in the precursor mixture to nano-scale particles. In one embodiment, the precursor mixture is sonicated for a time between about 5 minutes and about 2 hours. In another embodiment, the precursor mixture is sonicated for a time between about 10 minutes and about 1 hour. In still another embodiment, the precursor mixture is sonicated to for a time between about 15 minutes and about 30 minutes. In some embodiments, the precursor mixture is sonicated for about 5 minutes, 10 minutes, 15 minutes, 20 minutes, 25, minutes, 30 minutes, 35 minutes, 40 minutes, 45 minutes, 50 minutes, 55 minutes, 60 minutes, 90 minutes, or 120 minutes, or about 10 minutes, 15 minutes, 20 minutes, 25, minutes, 30 minutes, 35 minutes, 40 minutes, 45 minutes, 50 minutes, 55 minutes, or 60 minutes, or about 15 minutes, 20 minutes, 25, minutes, or 30 minutes.

As mentioned above, sonication is typically conducted at a low temperature in order to minimize the vapor pressure of the solvent. In this case, it is also advantageous to sonicate at a low temperature to avoid degrading the vitamin substance. As such, it can be advantageous to sonicate at a temperature in a range from about 0° C. to about 60° C., or in a range from about 1° C. to about 40° C., or in a range from about 2° C. to about 20° C., or in a range from about 5° C. to about 10° C.

In one embodiment, the sonicator transmits sonic energy into the precursor mixture in a sequence of pulses. For example, a typical sonication procedure sonication procedure calls for a pulse sequence of 5 seconds on/2 seconds off. If, for example, the sample is sonicated for a total of 21 minutes, during 15 minutes of that time the sample is undergoing active sonication.

The resulting nanoparticulate vitamin composition is stable and includes the liquid dispersion medium and the above-described particles. In some embodiments, the stabilized vitamin particles can be used to supplement dietary vitamin content in a number ways. For example, the dispersed particles can be directly combined with a number of foods including, but not limited to, water-based beverages, processed meat products, processed fish products, gels such as energy gels, jams, pastes, nutrition bars, bakery products, creams, sauces, dairy products, confections, or syrups, and combinations thereof.

In some embodiments, the stabilized vitamin particles can be purified from the solvent and used to supplement dietary vitamin content. For example, the stabilized vitamin particles can be purified from the solvent by filtration, centrifugation or by vitamin spray coating them onto sugar spheres or onto any one of the pharmaceutical excipients discussed above using, for example, a fluid-bed spray coater by techniques well known in the art. Purified nanoparticles can be directly combined with a number of foods including, but not limited to, pet foods, water-based beverages, processed meat products, processed fish products, gels such as energy gels, jams, pastes, nutrition bars, bakery products, creams, sauces, dairy products, confections, or syrups, and combinations thereof. In addition, the purified particles can be inserted into capsules or pressed into pills, caplets, or tablets and be used as vitamin supplements.

The stable nanoparticulate vitamin compositions may be embodied in other specific forms without departing from the spirit or essential characteristics of this disclosure. The described embodiments are to be considered in all respects only as illustrative and not restrictive. All changes that come within the meaning and range of equivalency of the claims are to be embraced within their scope.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method of preparing a stable nanoparticulate vitamin composition, comprising:
providing a precursor mixture that includes:
particles of at least one vitamin compound having a first size;
at least one solvent in which the at least one vitamin compound has a solubility of less than 10 mg/ml; and
molecules of at least one surface modifying agent; and
treating the precursor mixture to produce smaller sized vitamin particles having a second size in a range from about 1 nm to about 2000 nm,
wherein the molecules of the at least one surface modifying agent stably associate with and stabilize the smaller sized vitamin particles.

2. A method as recited in claim 1, wherein the particles of the at least one vitamin compound are provided as a powder or slurry of individual particles or agglomerates, wherein the first size is in a range of about 100 μm to about 2 μm.

3. A method as recited in claim 1, further comprising selecting at least one water-partitionable vitamin compound or at least one lipid-partitionable vitamin compound.

4. A method as recited in claim 3, wherein the at least one water-partitionable vitamin is selected from the group consisting of vitamin B₁, vitamin B₂, vitamin B₃, vitamin B₅, vitamin B₆, vitamin B₇, vitamin B₉, vitamin B₁₂, or vitamin C, and combinations thereof.

5. A method as recited in claim 3, wherein the at least one lipid-partitionable vitamin is selected from the group consisting of vitamin A, vitamin D, vitamin E, or vitamin K, and combinations thereof.

6. A method as recited in claim 1, wherein the solvent is selected from the group consisting of water, aqueous salt solutions, methanol, ethanol, propanol, butanol, glycerol, propylene glycol, propylene glycol ethers, dimethyl formamide, N-methyl pyrrolidone, acetone, diethyl ether, chloroform, benzene, tetrahydrofuran, hexanes, ethyl acetate, methyl methacrylate, toluene, phenyl ethers, vegetable oil, and combinations thereof.

7. A method as recited in claim 1, wherein the surface modifying agent includes at least one of an organic acid, a long-chain amine, a surfactant, an anionic surface stabilizer, a cationic surface stabilizer, a zwitterionic surface stabilizer, or an ionic surface stabilizer, and optionally includes one or more long-chain alcohols.

8. A method as recited in claim 7, wherein the organic acid is a fatty acid chosen from the group consisting of butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, uncosanoic acid, docosanoic acid, tricosanoic acid, tetracosanoic acid, undecylenic acid, myristoleic acid, palmitoleic acid, oleic acid, linoleic acid, alpha-linolenic acid, arachidonic acid, eicosapentaenoic acid, erucic acid, docosahexaenoic acid, and metals salts thereof.

9. A method as recited in claim 7, wherein the surface modifying agent includes at least one long-chain amine with a chain length of at least 6 carbon atoms.

10. A method as recited in claim 7, wherein the surfactant is selected from the group consisting of octylphenol ethoxylates, phosphonic acids, phosphinic acids, sulfonic acids, polyethylene glycol monoalkyl ethers, and combinations thereof.

11. A method as recited in claim 1, wherein the treating further comprises:
transferring the precursor mixture to a microfluidizer having an interaction chamber capable of producing one or more of shear, impact, cavitation, or attrition forces; and
subjecting the precursor mixture to said forces at a temperature not exceeding 60° C. and a fluid pressure of from about 3,000 to about 30,000 psi by passing the precursor mixture through said interaction chamber to obtain vitamin particles having an effective average particle size in a range from about 1 nm to about 2000 nm.

12. A method as recited in claim 1, wherein the treating further comprises:
sonicating the precursor mixture a temperature not exceeding 60° C., for a time in a range from about 5 minutes to about 2 hours,
wherein the sonicating suspends the particles of the at least one vitamin in the solvent allows the surface modifying agent to associate with the particles of the at least one vitamin, and
wherein the sonicating disrupts or breaks down the particles of the at least one vitamin into smaller vitamin particles having a second size in a range from about 1 nm to about 2000 nm.

13. A method as recited in claim 1, wherein vitamin particles of the stable, nanoparticulate vitamin composition are substantially sphere-shaped and/or substantially rod-shaped.

14. A method of preparing a stable nanoparticulate vitamin composition, comprising:
selecting at least one vitamin compound from a group consisting of water-partitionable vitamins or a group consisting of lipid-partitionable vitamins;
providing a precursor mixture that includes:
particles of at least one vitamin compound having a first size in a range of about 100 μm to about 2 μm, the particles being provided as a powder or slurry of individual particles or agglomerates;
at least one solvent in which the at least one vitamin compound has a solubility of less than 10 mg/ml; and
molecules of at least one surface modifying agent that are dispersible in the at least one solvent and are capable of stably bonding to the particles of the at least one vitamin compound; and
breaking down the particles of the at least one vitamin compound in the precursor mixture to produce the stable nanoparticulate vitamin composition by subjecting the precursor mixture to shear, impact, cavitation, sonication, and/or attrition forces,
wherein the stable nanoparticulate vitamin composition comprises smaller sized vitamin particles having a second size in a range from about 1 nm to about 2000 nm, and
wherein the molecules of the at least one surface modifying agent stably associate with and stabilize the smaller sized vitamin particles.

15. A method as recited in claim 14, wherein the at least one water-partitionable vitamin is selected from the group consisting of vitamin B₁, vitamin B₂, vitamin B₃, vitamin B₅, vitamin B₆, vitamin B₇, vitamin B₉, vitamin B₁₂, or vitamin C, and combinations thereof.

16. A method as recited in claim 14, wherein the at least one lipid-partitionable vitamin is selected from the group consisting of vitamin A, vitamin D, vitamin E, or vitamin K, and combinations thereof.

17. A method as recited in claim 14, wherein the solvent is selected from the group consisting of acetone, diethyl ether, chloroform, benzene, tetrahydrofuran, hexanes, ethyl acetate, methyl methacrylate, toluene, phenyl ethers, vegetable oils, and combinations thereof.

18. A method as recited in claim 14, wherein the solvent is selected from the group consisting of water, aqueous salt solutions, methanol, ethanol, propanol, butanol, glycerol, propylene glycol, or propylene glycol ethers, and combinations thereof.

19. A method as recited in claim 14, wherein the surface modifying agent includes at least one of an organic acid, a long-chain amine, a surfactant, an anionic surface stabilizer, a cationic surface stabilizer, a zwitterionic surface stabilizer, or an ionic surface stabilizer, and optionally includes one or more long-chain alcohols.

20. A stable nanoparticulate vitamin composition, comprising:
nano-particles of at least one vitamin compound having a size in a range from about 1 nm to about 2000 nm; and
molecules of at least one stabilizing agent associated with the nano-particles.

21. A stable nanoparticulate vitamin composition as recited in claim 20, wherein the nano-particles of the stable nanoparticulate vitamin composition have a size in a range from about 50 nm to about 1500 nm.

22. A stable nanoparticulate vitamin composition as recited in claim 20, wherein the nano-particles of the stable nanoparticulate vitamin composition have a size in a range from about 100 nm to about 1000 nm.

23. A stable nanoparticulate vitamin composition as recited in claim 20, wherein the nano-particles of at least one vitamin compound are selected from a group consisting of water-partitionable vitamins or a group consisting of lipid-partitionable vitamins.

24. A stable nanoparticulate vitamin composition as recited in claim 20, wherein the surface modifying agent includes at least one of an organic acid, a long-chain amine, a surfactant, an anionic surface stabilizer, a cationic surface stabilizer, a zwitterionic surface stabilizer, or an ionic surface stabilizer, and optionally includes one or more long-chain alcohols.

25. A stable nanoparticulate vitamin composition as recited in claim 20, further comprising at least one solvent in which the at least one vitamin compound has a solubility of less than 10 mg/ml, wherein the solvent is selected from the group consisting of water, aqueous salt solutions, methanol, ethanol, propanol, butanol, glycerol, propylene glycol, propylene glycol ethers, dimethyl formamide, N-methyl pyrrolidone, acetone, diethyl ether, chloroform, benzene, tetrahydrofuran, hexanes, ethyl acetate, methyl methacrylate, toluene, phenyl ethers, vegetable oil, and combinations thereof.
